SEQUENCE LISTING (8 sequences)

SEQ ID NO: 1 - DNA, 63 bp, synthetic construct
  source 1..63: mol_type = other DNA
atttatcacccgccatagtagacgtatcaccaggcagttgagacgaacattcctaagtctgaa

SEQ ID NO: 2 - DNA, 63 bp, synthetic construct
  source 1..63: mol_type = other DNA
acatgcgagggtccaataccgacgattacagcttgctacacgattcagacttaggaatgttcg

SEQ ID NO: 3 - DNA, 63 bp, synthetic construct
  source 1..63: mol_type = other DNA
actactatggcgggtgataaaacgtgtagcaagctgtaatcgacgggaagagcatgcccatcc

SEQ ID NO: 4 - DNA, 63 bp, synthetic construct
  source 1..63: mol_type = other DNA
acggtattggaccctcgcatgactcaactgcctggtgatacgaggatgggcatgctcttcccg

SEQ ID NO: 5 - DNA, 28 bp, synthetic construct
  source 1..28: mol_type = other DNA
catgcccctgtaatcgcccatgggtagc

SEQ ID NO: 6 - DNA, 92 bp, synthetic construct
  source 1..92: mol_type = other DNA
atttatcacccgccatagtagacgtatcaccaggcagttgagacgaacattcctaagtctgaaacatgcccctgtaatcgcccatgggtagc

SEQ ID NO: 7 - DNA, 76 bp, synthetic construct
  source 1..76: mol_type = other DNA
  misc_feature 76
acatgcgagggtccaataccgacgattacagcttgctacacgattcagacttaggaatgttcgaaaaaaaaaaaaa

SEQ ID NO: 8 - DNA, 63 bp, synthetic construct; identical to SEQ ID NO: 3
  source 1..63: mol_type = other DNA
  misc_feature 63

(19) United States
(12) Patent Application Publication
(10) Pub. No.: US 2025/0304639 A1
(43) Pub. Date: Oct. 2, 2025

(54) TETRAHEDRAL FRAMEWORK NUCLEIC ACID MODIFIED BY APTAMER AND TRANSFORMING GROWTH FACTOR beta 3, AND PREPARATION METHOD THEREOF

(71) Applicant: ZhuJiang Hospital of Southern Medical University, Guangzhou City (CN)

(72) Inventors: Zhaohua ZHU, Guangzhou City (CN); Xiaorui SHI, Guangzhou City (CN); Haowei CHEN, Guangzhou City (CN); Yang LI, Guangzhou City (CN); Song XUE, Guangzhou City (CN); Changhai DING, Guangzhou City (CN)

(21) Appl. No.: 18/679,608

(22) Filed: May 31, 2024

(30) Foreign Application Priority Data
Apr. 2, 2024 (CN) 202410401442.0

(51) Int. Cl. C07K 14/495; A61K 47/54; C12N 15/115

(52) U.S. Cl. CPC C07K 14/495; A61K 47/549; C12N 15/115; C12N 2310/16; C12N 2310/3513; C12N 2310/51

(57) ABSTRACT

The present disclosure provides a tetrahedral framework nucleic acid modified by aptamer and transforming growth factor β3 and a preparation method thereof, belonging to the field of biomedicine. The tetrahedral framework nucleic acid modified by aptamer and transforming growth factor β3 combines transforming growth factor β3 through disulfide bonds, and sequences of four DNA single strands of the tetrahedral framework nucleic acid are shown in SEQ ID NO: 1-4, one of the four DNA single strands of the tetrahedral framework nucleic acid also comprises an extended aptamer sequence, and the extended aptamer sequence is shown in SEQ ID NO: 5.

### CROSS-REFERENCE TO RELATED APPLICATIONS

This application claims priority to Chinese Patent Application No. 202410401442.0, filed on Apr. 2, 2024, the contents of which are hereby incorporated by reference.

### INCORPORATION BY REFERENCE STATEMENT

This statement, made under Rules 77(b)(5)(ii) and any other applicable rule incorporates into the present specification of an XML file for a “Sequence Listing XML” (see Rule 831(a)), submitted via the USPTO patent electronic filing system or on one or more read-only optical discs (see Rule 1.52(e)(8)), identifying the names of each file, the date of creation of each file, and the size of each file in bytes as follows:
File name: SequenceListing347207_2024-4083.xml
Creation date: 29 May 2024
Byte size: 8,728

### TECHNICAL FIELD

The present disclosure relates to the field of biomedicine, and in particular to a tetrahedral framework nucleic acid modified by an aptamer and a transforming growth factor β3 (TGF-β3), and a preparation method thereof.

### BACKGROUND

Osteoarthritis (OA) is the most common joint disease in the elderly population, with joint pain, stiffness, and limited mobility as the main symptoms, imposing a huge disease burden on the patient's family and society. However, there is still a lack of effective treatments for OA. Numerous studies have shown that degeneration and destruction of articular cartilage are major pathological changes in the progression of OA, and therefore promoting regeneration and formation of cartilage is one of the potential treatments for OA. One of the current strategies to promote cartilage regeneration is to use transforming growth factor beta 3 (β3) in vitro to induce chondrogenic differentiation of mesenchymal stem cells (MSCs) followed by intra-articular injections. Attempts are being made to find breakthrough treatments for OA through stem cell therapy, and although a large number of clinical trials have been conducted, no favourable results have been reported for stem cell therapy, which may be related to the immaturity of the technology of stem cell therapy.

The stem cell therapy mainly adopts the exogenous stem cell injection strategy at present, and such exogenously injected stem cells not only require a tedious preparation process, but also pose a great challenge in terms of their survival after successful preparation and injection into the joint cavity. Specifically, MSCs from different sources have varied differentiation abilities, different clinical efficacy and culture characteristics. Therefore, an important consideration for the success of MSCs therapy is to select an appropriate cell source, with common sources including bone marrow, adipose, synovial fluid, and synovial membrane. Furthermore, the in vitro culture conditions of such screened MSCs are challenging and extremely costly, and their survival rate is not guaranteed after being introduced into the body. In contrast, the direct mobilization of intra-articular MSCs by joint cavity injection of differentiation inducers faces a series of limitations such as high efficiency of joint clearance of inducers alone and lack of MSCs targeting. Therefore, although stem cell therapy is considered to be the third revolution in medical history in the 21st century, there are still many shortcomings in existing stem cell therapies that need to be improved before clinical transformation may be achieved.

### SUMMARY

The objectives of the present disclosure is to provide a tetrahedral framework nucleic acid modified by aptamer and transforming growth factor β3 (TGF-β3), and preparation method and application thereof, so as to solve the problems existing in the prior art; by delivering drugs to targeted bone marrow mesenchymal stem cells, the biological efficacy and intra-articular utilisation of TGF-β3 is increased, thereby effectively protecting osteoarthritis (OA) chondrocytes and ameliorating OA pain symptoms and disease progression.

In order to achieve the above objectives, the present disclosure provides the following technical schemes.

The present disclosure provides a tetrahedral framework nucleic acid modified by aptamer and transforming growth factor β3, where the tetrahedral framework nucleic acid is obtained by combining transforming growth factor β3 through disulfide bonds, and sequences of four DNA single strands of the tetrahedral framework nucleic acid are shown in SEQ ID NO: 1-4, one of the four DNA single strands of the tetrahedral framework nucleic acid also includes an extended aptamer sequence, and the extended aptamer sequence is shown in SEQ ID NO: 5.

Optionally, the tetrahedral framework nucleic acid is a sulfhydryl modified tetrahedral framework nucleic acid.

The present disclosure also provides a preparation method of the tetrahedral framework nucleic acid modified by the aptamer and the transforming growth factor β3, including the following steps:
assembling four DNA single strands by self-assembly to synthesize a tetrahedral framework nucleic acid;
subjecting the transforming growth factor β3 and N-succinimidyl-3-(2-pyridyl dithio)propionate (SPDP) to a mixing reaction to obtain activated transforming growth factor β3; and
subjecting the tetrahedral framework nucleic acid and the activated transforming growth factor β3 to a mixing reaction to obtain the tetrahedral framework nucleic acid modified by the aptamer and the transforming growth factor β3.

Optionally, conditions for the self-assembly include: denaturation at 95 degrees Celsius (C) for 10 minutes (min), and cooling at 4° C. for 20-30 min.

Optionally, a volume ratio of the transforming growth factor β3 to the N-succinimidyl-3-(2-pyridyl dithio)propionate is 1: (1-3); and
conditions for the mixing reaction of the transforming growth factor β3 to the N-succinimidyl-3-(2-pyridyl dithio)propionate include: reacting at 20-25° C. for 2-3 hours (h).

Optionally, a volume ratio of the tetrahedral framework to the activated transforming growth factor β3 is (1-2): 1; and
conditions for the mixing reaction of the tetrahedral framework and the activated transforming growth factor β3 include: reacting at 20-25° C. for 2-3 h.

The present disclosure also provides an application of the tetrahedral framework nucleic acid modified by the aptamer and the transforming growth factor β3 in any one of the following:
(1) application in preparing medicines for promoting bone marrow mesenchymal stem cells in terms of proliferation;
(2) application in preparing medicines for inducing bone marrow mesenchymal stem cells to differentiate into chondrocytes;
(3) application in preparing medicines for protecting osteoarthritis cartilage;
(4) application in preparing medicines for treating osteoarthritis; and
(5) application in preparing medicines for improving therapeutic effects of transforming growth factor β3 on osteoarthritis.

The present disclosure also provides a medicine for treating osteoarthritis, including the medicine for treating osteoarthritis.

The present disclosure achieves the following technical effects.

In the present disclosure, HM69-tetrahedral framework nucleic acid (H-TFNA) is synthesised and HM69-TFNA@TGF-β3 complex (HTT) is formed by binding TGF-β3 via SPDP reaction. The HTT is tested in vitro to be biostable and has a good biological function to promote the proliferation of MSCs in vitro; it also has a good capability of inducing the differentiation of MSCs into chondrocytes, with enhanced protection of OA chondrocytes. In addition, the results of in vivo tests also show that HTT has a good effect on relieving OA pain, improving OA joint function and protecting OA cartilage.

The present disclosure synthesize the HM69-TFNA@TGF-β3 drug-carrying system innovatively, which improves the bioefficacy and intra-articular utilisation of TGF-β3 by targeting bone marrow mesenchymal stem cells for drug delivery, and effectively protects OA chondrocytes and improves the pain symptoms and disease progression of OA.

### BRIEF DESCRIPTION OF THE DRAWINGS

In order to explain the embodiments of the present disclosure or the technical scheme in the prior art more clearly, the drawings needed in the embodiments are briefly introduced below. Obviously, the drawings described below are only some embodiments of the present disclosure, and other drawings may be obtained according to these drawings without creative work for ordinary people in the field.

FIG. 1 is a schematic diagram of four DNA single strands and TGF-β3 synthesizing functional HTT.

FIG. 2A shows a pattern diagram of activated TGF-β3 synthesis.

FIG. 2B shows a pattern diagram of synthesizing TFNA or H-TFNA.

FIG. 2C shows a pattern diagram of HTT synthesis.

FIG. 3 shows an AFM of the material.

FIG. 4A shows the PAGE test results of the successful synthesis of HTT.

FIG. 4B shows the in vitro stability test results of the successful synthesis of HTT.

FIG. 5A shows the measurement results of Zeta potential of HTT.

FIG. 5B shows the measurement results of molecular particle size of HTT.

FIG. 6A is a fluorescence chart showing the ability of immunofluorescence to detect the entry of HTT, TTC and sDTC into bone marrow mesenchymal stem cells.

FIG. 6B is a statistical chart showing the ability of immunofluorescence to detect the entry of HTT, TTC and sDTC into bone marrow mesenchymal stem cells.

FIG. 7 shows the results of cell proliferation detected by CCK-8 method.

FIG. 8A the results of cell cycle detection in the treatment and control groups.

FIG. 8B shows a statistical chart of the data in FIG. 8A.

FIG. 9A shows protein expression of Aggrecan, Collagen II, β-catenin and SOX-9 in different groups of mesenchymal stem cells at day 21.

FIG. 9B shows gene expression of Aggrecan at 7 days in different groups of mesenchymal stem cells, where data are expressed as mean±SD (n=3).

FIG. 9C shows gene expression of Collagen II at 7 days in different groups of mesenchymal stem cells, where data are expressed as mean±SD (n=3).

FIG. 9D shows gene expression of SOX-9 at 7 days in different groups of mesenchymal stem cells, where data are expressed as mean±SD (n=3).

FIG. 9E shows quantification of Aggrecan protein expression levels.

FIG. 9F shows quantification of Collagen II protein expression levels.

FIG. 9G shows quantification of β-catenin protein expression levels.

FIG. 9H shows quantification of SOX-9 protein expression levels.

FIG. 9I shows glycosaminoglycan (GAG) deposition results (shown by Alisin blue staining) in bone marrow mesenchymal stem cells at day 14 (Inserted images: overall photographs of the culture wells).

FIG. 9J shows immunohistochemical staining results of type II collagen in cell spheres induced by different bone marrow mesenchymal stem cells; statistical analysis: *p<0.05, **p<0.01, ***p<0.001, ****p<0.0001, ns indicates no significant difference.

FIG. 10A is a schematic diagram of the transwell test.

FIG. 10B shows protein expression of Collagen II, MMP-3 and MMP-13 in OA chondrocytes after 24 h of co-culture with different BMSCs.

FIG. 10C shows quantification of Collagen II protein expression levels, and data are expressed as mean±standard deviation (n=3). Statistical analysis: *p<0.05, **p<0.01, ***p<0.001, ****p<0.0001, and ns indicates no significant difference.

FIG. 10D shows quantification of MMP-3 protein expression levels, and data are expressed as mean±standard deviation (n=3). Statistical analysis: *p<0.05, **p<0.01, ***p<0.001, ****p<0.0001, and ns indicates no significant difference.

FIG. 10E shows quantification of MMP-13 protein expression levels, and data are expressed as mean±standard deviation (n=3). Statistical analysis: *p<0.05, **p <0.01, ***p<0.001, ****p<0.0001, and ns indicates no significant difference.

FIG. 11A shows the tests at various time points in animal experiments.

FIG. 11B is a schematic diagram of DMM operation and treatment.

FIG. 11C shows in vivo imaging of mice after HHT injection.

FIG. 11D shows representative images of gait analysis in 8-week DMM mice.

FIG. 11E shows the quantitative analysis of RH area compared with LH at weeks 2, 4, 6, and 8 after sham operation or DMM; statistical analysis: *p<0.05, **p<0.01, ***p<0.001, and ns denotes no significant difference.

FIG. 11F shows the quantitative analysis of RH stress at weeks 2, 4, 6, and 8 after sham operation or DMM compared with LH; statistical analysis: *p<0.05, **p<0.01, ***p<0.001, and ns denotes no significant difference.

FIG. 11G shows the quantification of RH oscillation velocity at weeks 2, 4, 6, and 8 after sham operation or DMM compared with LH; statistical analysis: *p<0.05, **p<0.01, ***p<0.001, and ns denotes no significant difference.

FIG. 12A shows the representative plots of Safranin-O/Fast green staining of knee joints after different treatments.

FIG. 12B shows the OARSI scores measured separately based on histological analysis of samples collected at 6 weeks postoperatively; statistical analysis: *p<0.05, **p<0.01, ***p<0.001, ****p<0.0001.

FIG. 12C shows the osteoarthritis research society international (OARSI) scores measured separately based on histological analysis of samples collected at 10 weeks postoperatively; statistical analysis: *p<0.05, **p<0.01, ***p<0.001, ****p<0.0001.

FIG. 12D shows the representative plots of toluidine blue staining of knee joints after different treatments.

FIG. 12E illustrates the measurement of cartilage area based on histological analysis of specimens collected at 6 and 10 weeks postoperatively; statistical analysis: *p<0.05, **p<0.01, ***p<0.001, ****p<0.0001.

FIG. 13A shows the immunohistochemical staining results of the knee joint after different treatments.

FIG. 13B shows the semiquantitative analysis of type II collagen expression in immunohistochemical staining, where data are expressed as mean±standard deviation (n=3). statistical analysis: *p<0.05, **p<0.01, ***p<0.001, ****p<0.0001.

FIG. 13C shows the semiquantitative analysis of aggregated proteins in immunohistochemical staining, where data are expressed as mean±standard deviation (n=3). statistical analysis: *p<0.05, **p<0.01, ***p<0.001, ****p<0.0001.

FIG. 13D illustrates the immunohistochemical staining (MMP-3 and MMP-13) results of the knee joint after different treatments.

FIG. 13E shows the semiquantitative analysis of MMP-3 expression in IHC staining; data are expressed as mean±standard deviation (n=3). statistical analysis: *p<0.05, **p<0.01, ***p<0.001, ****p<0.0001.

FIG. 13F shows the semiquantitative analysis of MMP-13 expression in IHC staining; data are expressed as mean±standard deviation (n=3). statistical analysis: *p<0.05, **p<0.01, ***p<0.001, ****p<0.0001.

FIG. 13G shows the TUNEL staining of the knee joint after different treatments.

FIG. 13H shows the semi-quantitative analysis of TUNEL-positive cells in TUNEL staining; data are expressed as mean±SD (n=3); statistical analysis: *p<0.05, **p<0.01, ***p<0.001, ****p<0.0001.

### DETAILED DESCRIPTION OF THE EMBODIMENTS

A number of exemplary embodiments of the present disclosure are now described in detail, and this detailed description should not be considered as a limitation of the present disclosure, but should be understood as a rather detailed description of certain aspects, characteristics and embodiments of the present disclosure.

It should be understood that the terminology described in the present disclosure is only for describing specific embodiments and is not used to limit the present disclosure. In addition, for the numerical range in the present disclosure, it should be understood that each intermediate value between the upper limit and the lower limit of the range is also specifically disclosed. Intermediate values within any stated value or stated range, as well as each smaller range between any other stated value or intermediate values within the stated range are also included in the present disclosure. The upper and lower limits of these smaller ranges can be independently included or excluded from the range.

Unless otherwise specified, all technical and scientific terms used herein have the same meaning as commonly understood by one of ordinary skill in the art to which this disclosure relates. Although the present disclosure only describes the preferred methods and materials, any methods and materials similar or equivalent to those described herein can also be used in the practice or testing of the present disclosure. All documents mentioned in this specification are incorporated by reference to disclose and describe methods and/or materials related to the documents. In case of conflict with any incorporated document, the contents of this specification shall prevail.

It is obvious to those skilled in the art that many improvements and changes can be made to the specific embodiments of the present disclosure without departing from the scope or spirit of the present disclosure. Other embodiments will be apparent to the skilled person from the description of the disclosure. The description and embodiments of that present disclosure are exemplary only.

The terms “including”, “comprising”, “having” and “containing” used in this specification are all open terms, which means including but not limited to.

TGF-β3 is a commonly used substance to induce chondrogenesis of stem cells in vitro, mainly through the significant expansion of chondroprogenitor cells. It is used as the most common standard additive and encapsulated in various scaffolds to induce chondrogenesis of MSCs. However, low-dose injection of TGF-β3 will be quickly eliminated by the metabolism of joint fluid, and high-dose application of TGF-β3 will lead to chondrocyte hypertrophy and even inhibit chondrogenesis, and the in vivo application is therefore limited. Tetrahedral framework nucleic acid (TFNA) is a highly cell-friendly DNA nanomaterial widely reported for the treatment of various inflammatory diseases. Accordingly, in order to improve existing stem cell therapies for stimulating the differentiation of MSCs in situ within the joints, the present disclosure combines TFNA with TGF-β3 for targeting and precisely regulating the differentiation of MSCs. To make the binding more stable, the present disclosure also modifies the sulfhydryl group on TFNA and binds TFNA to TGF-β3 via disulfide bonds. A special DNA sequence HM69 is extended on TFNA, an adapter for targeting stem cells, so as to more accurately recognize stem cells and deliver TGF-β3. The complex formed by the present disclosure is composed of HM69, TFNA and TGF-β3 (referred to as HHT), and also retains the original advantages of the three and contributes synergistically to the chondrogenic differentiation of MSCs. Specific embodiments are further described below by way of example.

### Embodiment 1

### 1. Preparation Method of HTT

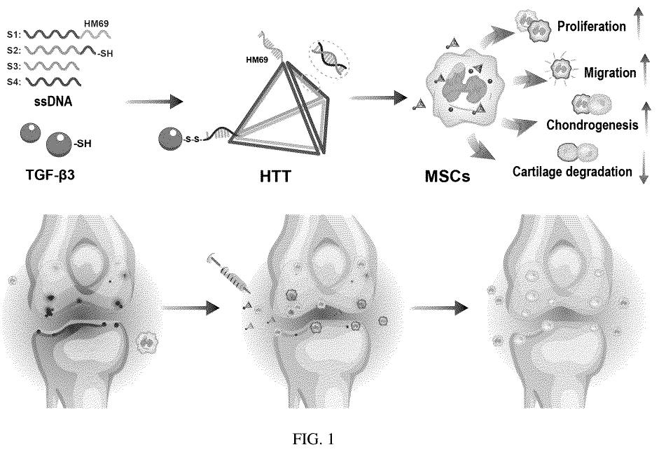

(1) TFNA is prepared on the basis of previous research, and the preparation method (as shown in FIG. 1) is as follows: four pre-designed and synthesized ssDNA sequences (see Table 1) stored at −20° C. are centrifuged at 10000 g at 4° C. for 10 min, and dissolved in water without DNAse until the concentration is 100 μM; next, 1 microliter (μL) of each ssDNA with a concentration of 100 μM is added to a 200 μL centrifuge tube, where the centrifuge tube includes 96 μL of TM buffer solution containing 10 mM Tris-HCl and 50 mM MgCl₂ (pH=8.0); after passing through a gentle vortex mixing tube, the mixture is denatured at 95° C. for 10 min, and then rapidly cooled at 4° C. for 30 min. Finally, the solution is kept in the thermal cycler for 20 min to maintain the tetrahedron structure and obtain TFNA.

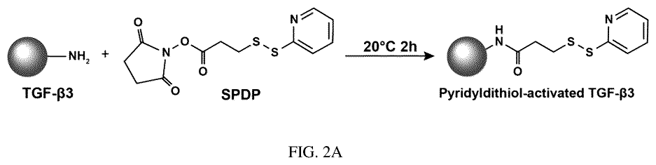

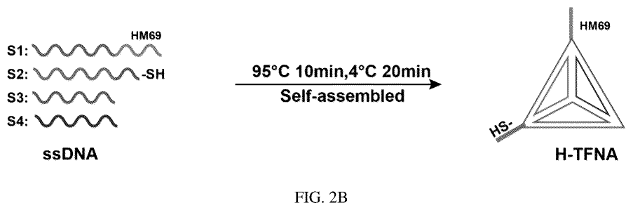

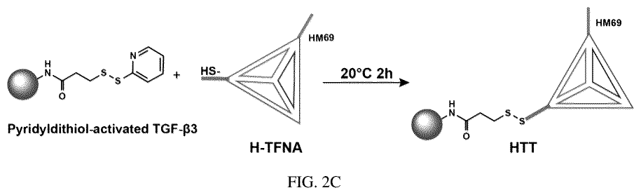

In the same method, four ssDNA sequences are replaced by four ssDNA sequences as shown in Table 2 to synthesize H-TFNA (see FIG. 2B for the synthetic pattern).

(2) N-succinimidyl-3-(2-pyridyl dithio)propionate (SPDP) is prepared into a solution with a stock solution concentration of 10 mmol/L, and TGF is prepared into a solution with a concentration of 10 nmol/L. After the stock solution of SPDP is diluted 1000 times, a working solution with a concentration of 10 nmol/L is prepared.

The SPDP working solution and TGF-β3 solution are mixed according to the volume ratio of 3:1, and reacted for 2 h at room temperature, and then centrifuged at 12,000 g for 15 min at 4° C. to remove the reaction by-product and the excess unreacted SPDP reagent mixed in the SPDP modified TGF-β3, and the activated TGF-β3 is obtained (see FIG. 2A for the synthetic pattern).

(3) H-TFNA solution is added to SPDP-modified TGF-β3 solution at a volume ratio of 1:1 and the reaction mixture is incubated overnight at room temperature; the reaction mixture is successfully coupled to obtain HM69-TFNA@TGF-β3 (HTT, the synthetic pattern is shown in FIG. 2C), and purified by centrifugation at 12,000 g for 15 min at 4° C.

The sequences of TFNA are shown in Table 1 below.

TABLE 1

| S/N | Sequence (5′-3′) |
|---|---|
| S1 | ATTTATCACCCGCCATAGTAGACGTATCAC |
| | CAGGCAGTTGAGACGAACATTCCTAAGTCT |
| | GAA |
| | (SEQ ID NO: 1) |
| S2 | ACATGCGAGGGTCCAATACCGACGATTACA |
| | GCTTGCTACACGATTCAGACTTAGGAATGT |
| | TCG |
| | (SEQ ID NO: 2) |
| S3 | ACTACTATGGCGGGTGATAAAACGTGTAGC |
| | AAGCTGTAATCGACGGGAAGAGCATGCCCA |
| | TCC |
| | (SEQ ID NO: 3) |
| S4 | ACGGTATTGGACCCTCGCATGACTCAACTG |
| | CCTGGTGATACGAGGATGGGCATGCTCTTC |
| | CCG |
| | (SEQ ID NO: 4) |

The sequences of H-TFNA are shown in Table 2 below.

TABLE 2

| | Sequence |
|---|---|
| S/N | (5′-3′) |
| S1* | ATTTATCACCCGCCATAGTAGACGTATCAC |
| | CAGGCAGTTGAGACGAACATTCCTAAGTCT |
| | GAAACATGCCCCTGTAATCGCCCATGGGTA |
| | GC |
| | (SEQ ID NO: 6) |
| S2* | ACATGCGAGGGTCCAATACCGACGATTACA |
| | GCTTGCTACACGATTCAGACTTAGGAATGT |
| | TCGAAAAAAAAAAAAA-SH |
| | (SEQ ID NO: 7) |
| S3* | ACTACTATGGCGGGTGATAAAACGTGTAGC |
| | AAGCTGTAATCGACGGGAAGAGCATGCCCA |
| | TCC-CY5 |
| | (SEQ ID NO: 8) |
| S4 | ACGGTATTGGACCCTCGCATGACTCAACTG |
| | CCTGGTGATACGAGGATGGGCATGCTCTTC |
| | CCG |
| | (SEQ ID NO: 4) |
| HM69 | CATGCCCCTGTAATCGCCCATGGGTAGC |
| | (SEQ ID NO: 5) |

The sequences shown in Table 1 and Table 2 are all synthesized by Guangzhou IGE Biotechnology Co., Ltd.

### 2. Characterization of Successful Synthesis of HTT

### 2.1 Atomic Force Microscopy (AFM) to Observe the Surface Morphology of HTT

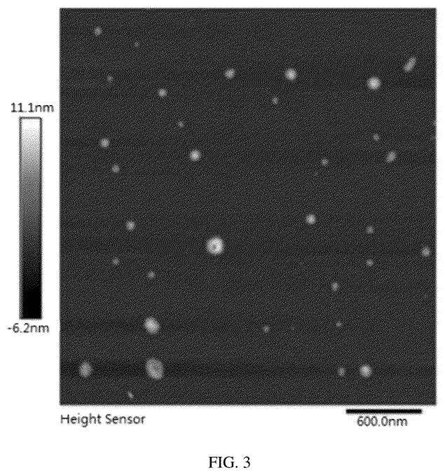

100 μL of HTT solution with 1 μM concentration is prepared, and an appropriate amount of the solution is taken onto the AFM detection plate and photographed under the microscope. The results are shown in FIG. 3 and the height is found to be 5.5 nm.

### 2.2 PAGE Nucleic Acid Electrophoresis for Detecting Molecular Weight of HTT

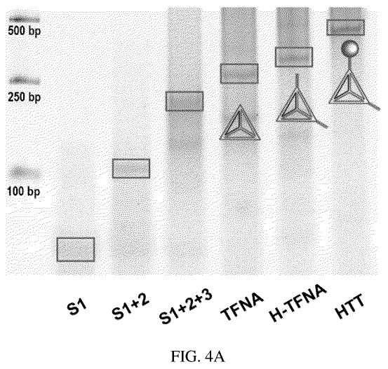

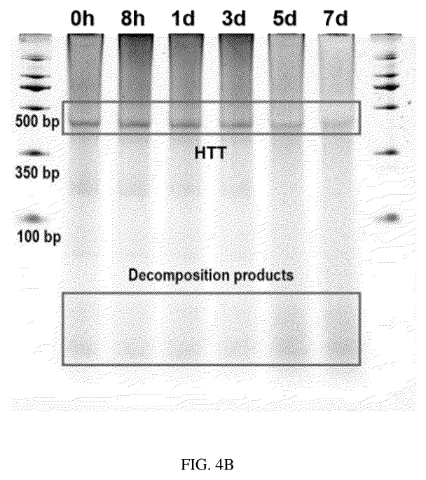

100 μL of single-stranded DNA solution (S1) with a concentration of 1 μM, 2-stranded DNA mixed solution (S1+S2), 3-stranded DNA mixed solution (S1+S2+S3), TFNA solution alone (TFNA), TFNA solution loaded with HM69 (H-TFNA), and HTT solution (HTT) are prepared. All the solutions are denatured at 95° C. for 10 min, then rapidly cooled at 4° C. for 30 min. Finally, the solutions are kept in a thermal cycler for 20 min, and then added with 20 μL of 6xDNA loading buffer. Separately, 30 μL from each solution is added to the PAGE gel lanes and electrophoresed at 120 volts (V) for 30-40 min, and the development results are shown in FIG. 4A. The results reveal that successfully formed HTT and other ssDNA assemblies are separated based on their mobility. It is observed that HTT moves slower than the others with bands positioned higher up, indicating that it has the highest molecular weight, meaning that HTT is successfully synthesised.

### 2.3 Dynamic Light Scattering (DLS) for Detecting the Particle Size and Zeta Potential of HTT

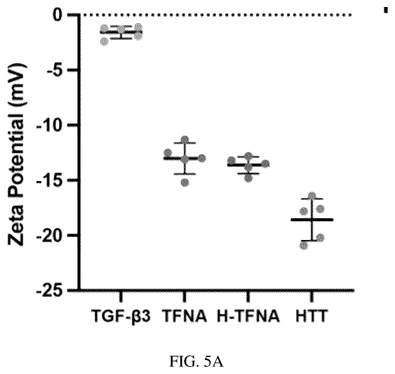

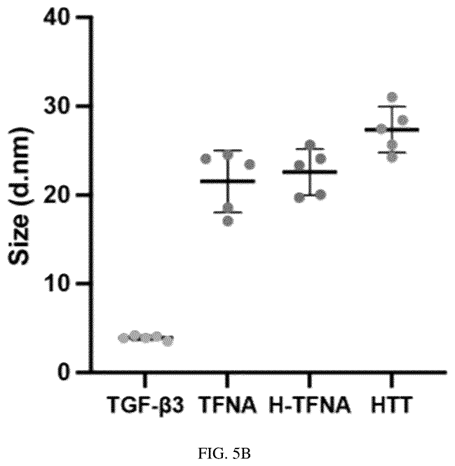

1 milliliter (mL) of TGF-β3 solution, TFNA solution, H-TFNA solution, and HTT solution at a concentration of 1 μM are prepared. Firstly, an appropriate amount of pure water is taken into the DLS test tube and the test is switched on to clean the instrument. After that, the substances in each group of solutions are detected separately. The results are shown in Table 3 and FIG. 5A and FIG. 5B.

TABLE 3

| Samples | Potential (mV) | Size (d · nm) |
|---|---|---|
| TGF-β3 | −1.58 ± 0.55 | 3.92 ± 0.24 |
| TGF-β3 | −13.02 ± 1.41 | 21.53 ± 3.48 |
| H-TFNA | −13.62 ± 0.76 | 22.57 ± 2.60 |
| HTT | −18.58 ± 1.89 | 27.17 ± 2.26 |

### 2.4 Determination of Stability of HTT in Vitro

In order to understand the in vitro stability of HTT, a time gradient is set up for the time points of 0 h, 8 h, 1 day, 3 day, 5 day and 7 day after HTT synthesis, and polyacrylamide gel electrophoresis (PAGE) is performed on the 7th day under the conditions as previously described, and the HTT decomposition is judged by observing the colour intensity of the final band, and lighter colours of the electrophoretic bands at the same concentration indicate that the more the material is decomposed.

The results are shown in FIG. 4B, where the initial concentration of HTT in each lane is 1 nM, and the bands become lighter and lighter in colour with time, indicating that the concentration of HTT is getting lower and lower, and the decomposition is increasing. The lightest colour occurs at day 7, whereas the colour of the small molecule DNA in the lower layer of the gel becomes darker and darker, confirming that the material is gradually decomposing over time.

### Embodiment 2 Effect Verification

### 1. Stem cell Uptake HTT

HTT, TFNA@TGF-β3 complex (TTC) and ssDNAs@TGF-β3 complex (sDTC) (CY5-HTT, CY5-TTC, CY5-sDTC) loaded with CY5 are added to bone marrow mesenchymal stem cells to confirm the interaction effect between HTT and bone marrow mesenchymal stem cells. Bone marrow mesenchymal stem cells are seeded on a 24-well cell slide at a density of 1×10⁴ cells per well and cultured in normal growth medium for 1 day. Cells are cultured with CY5-HTT (250 nM), CY5-TTC (250 nM) or CY5-sDTC (250 nM) in fresh DMEM/F12 containing 1% FBS, with 3 replicates in each group. After 12 h incubation, the cell samples are fixed with 4% paraformaldehyde for 30 min and infiltrated with 0.5% Triton X-100 for 10 min. Then, the cytoskeleton is stained with FITC labeled phalloidin for 30 min. Then the nucleus is stained with 4′,6-diamino-2-phenylindole (DAPI) for 10 min. Finally, the images of all samples are captured by fluorescence microscope.

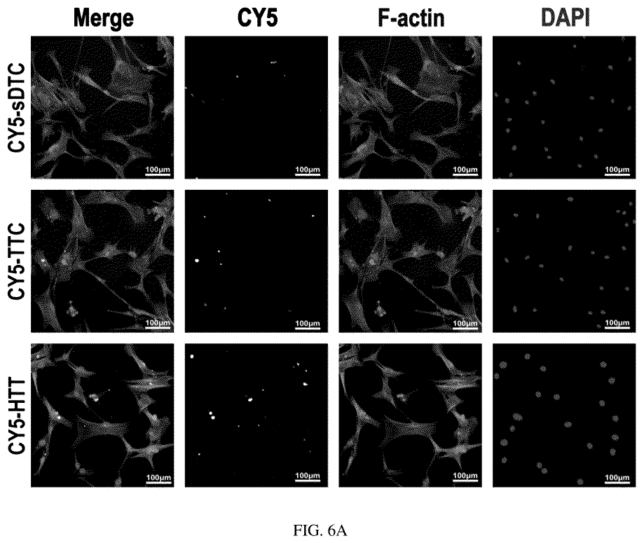

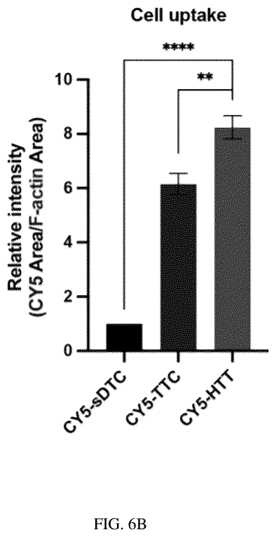

The results are shown in FIG. 6A-FIG. 6B, where it is found that HTT is more internalised into normal bone marrow mesenchymal stem cells compared to other subgroups after visualisation of the statistics.

### 2. Determination of Cell Counting Kit-8 (CCK-8)

Bone marrow mesenchymal stem cells are inoculated into 96-well plates and co-cultured with HTT at different concentrations (0, 62.5, 125, 250 and 375 nM). After 24 h incubation, the cytotoxicity of HTT is detected by standard CCK-8 method (APExBio, USA).

The formula for calculating cell viability is: cell viability (%)=(ODₛₐₘₚₗₑ−OD_{cck})/(OD_{control}−ODCCK)×100%.

Among them, ODₛₐₘₚₗₑ is the OD value of MSCs co-cultured with HTT, OD_{control} is the OD value of pure MSCs, and OD_{cck} is the OD value of culture medium containing CCK-8 solution.

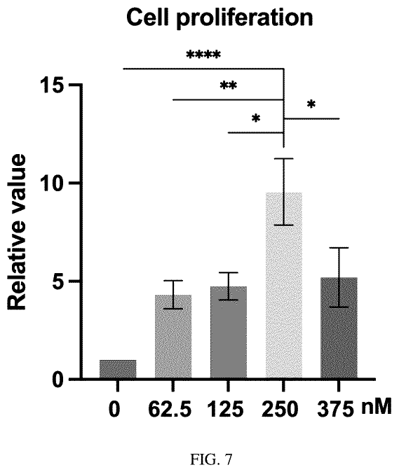

As shown in FIG. 7, the results of CCK8 cell activity test show that the cell activity of bone marrow mesenchymal stem cells in the HTT treatment group is higher than that in the control group, indicating that HTT promotes the proliferation of bone marrow mesenchymal stem cells, and the 250 nM concentration of HTT offers the best effect on the proliferation of bone marrow mesenchymal stem cells. Therefore, 250 nM is selected as the optimal concentration of HTT, and bone marrow mesenchymal stem cells are treated with this concentration, and all subsequent cell experiments are carried out.

### 3. Cell Cycle Determination

Cell cycle changes are analyzed by flow cytometry. In short, primary articular chondrocytes are cultured in normal DMEM medium for 24 h, and then further cultured with 250 nM HTT or serum-free medium at 37° C. After exposure to HTT for 24 h, chondrocytes are harvested, rinsed with PBS for three times, and the treated cells are fixed in 4% paraformaldehyde cold solution at 4° C. overnight. Subsequently, chondrocytes are digested with RNase for 30 min, resuspended with propidium iodide staining solution, and incubated in the dark at 37° C. for 1 h. The distribution changes of G1, S and G2 phases are detected by flow cytometry (FC500 Beckman, IL, USA), and analyzed by WinMDI2.9 and WinCycle software.

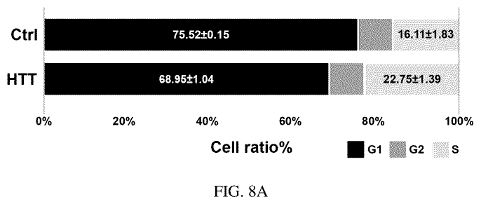

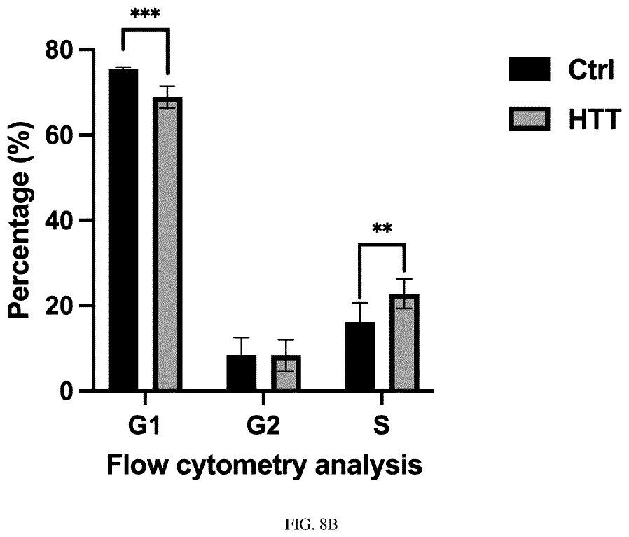

As shown in FIG. 8A-FIG. 8B, the cell flow results indicate that the proportion of bone marrow mesenchymal stem cells in the HTT-treated group is significantly higher than that of the control group in the S-phase, while the proportion of cells in the G1-phase decreases. Thus, it is demonstrated in the present disclosure that HTT promotes the proliferation of bone marrow mesenchymal stem cells by regulating the cell cycle.

### 4. Promotion of Cartilage Formation by HTT in Bone Marrow Mesenchymal Stem Cells

Bone marrow MSCs from different groups are cultured with chondrogenic medium of Cyagen Biosciences for 7 days and 21 days, respectively. RT-qPCR is performed to analyse the expression levels of chondrogenic genes, such as SRY-box transcription factor 9 (SOX-9), aggrecan and type II collagen, at 7 d. The expression levels of these genes are detected by western blot at 21 d. In addition, MSCs from different groups are inoculated into 12-well plates for 14 days as described above. In addition, different groups of MSCs are inoculated in 12-well plates and subjected to chondrogenic induction for 14 days as described above. Glycosaminoglycan (GAG) formation is detected by Alisin blue staining.

Mesenchymal stem cells are further induced into cell particles according to the manufacturer's instructions. Briefly, bone marrow mesenchymal stem cells (4×10⁵ cells) are suspended in a 15 mL centrifuge tube. After centrifugation of 250 g for 4 min, the supernatant is removed, and MSCs chondrogenesis medium (muxx-90041, Cyagen Biosciences) is slowly added to the tube without resuspending the cells. After incubation for another 2 days, cell microspheres are formed at the bottom of the test tube. Cartilage medium is changed every three days. After incubation for 4 weeks, the cells are fixed, embedded in paraffin and sliced. Immunohistochemistry (IHC) is used to detect the expression of type II collagen.

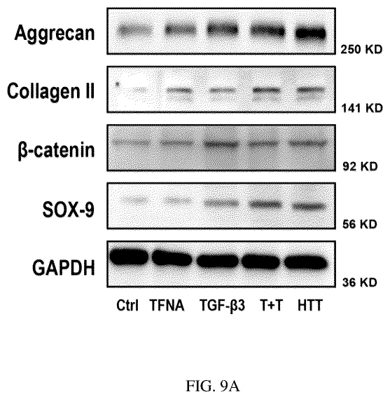

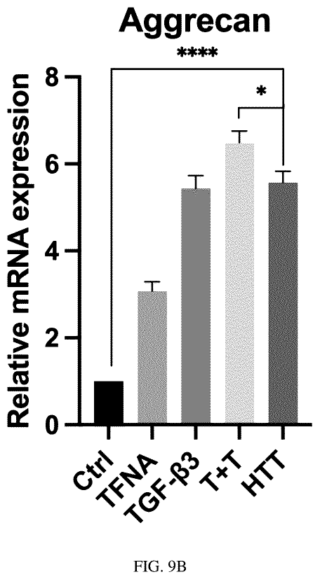

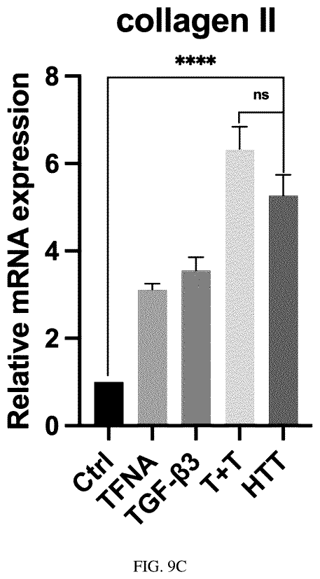

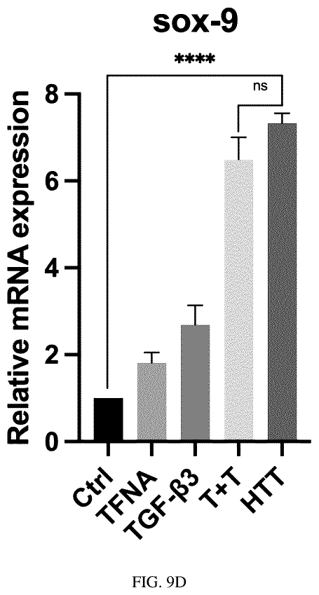

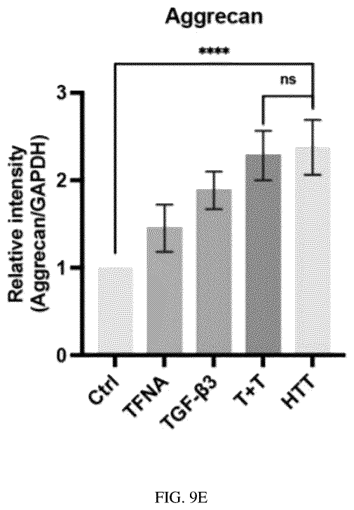

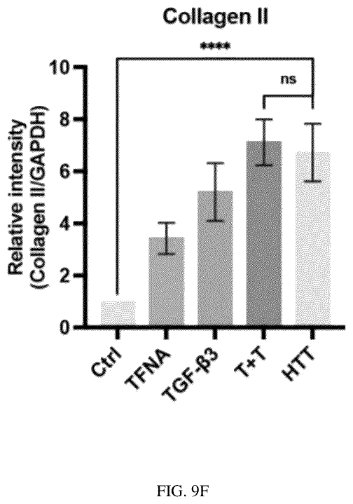

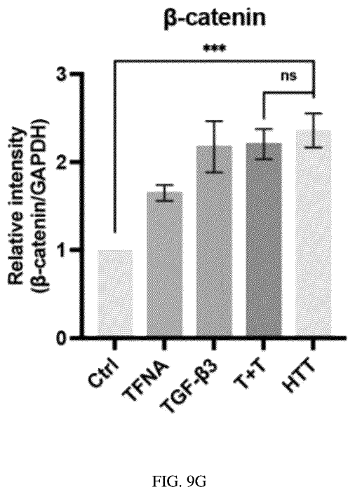

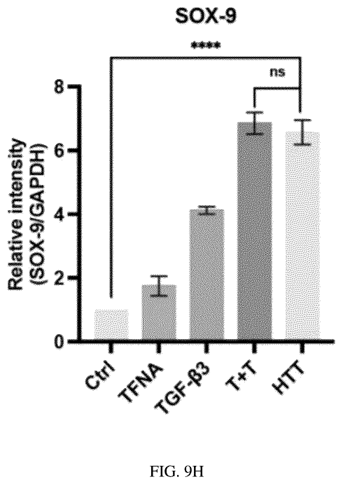

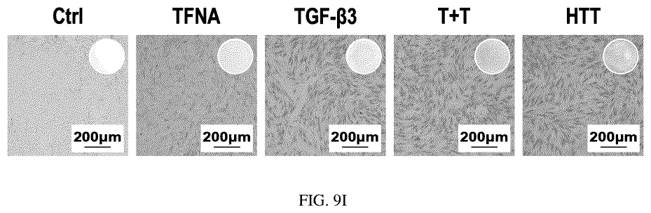

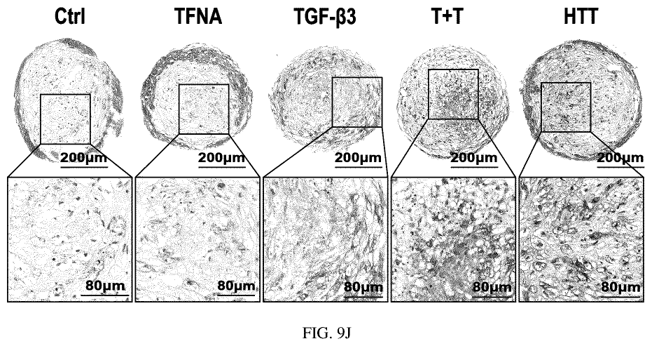

The results are illustrated in FIG. 9A, FIG. 9B, FIG. 9C, FIG. 9D, FIG. 9E, FIG. 9F, FIG. 9G, FIG. 9H, FIG. 9I, and FIG. 9J. 7 days after the induction of chondrogenesis, the mRNA expression levels of chondrogenic markers SOX-9, aggregation protein (Aggrecan), and type II collagen (Collagen II) are significantly higher in the HTT-treated group compared with the PBS-treated group; moreover, the expression level of HTT-treated group is higher than that of TGF-β3 or TFNA treatment group and similar to that of T+T treatment group (FIG. 9B). After 21 days of chondrogenesis, the expression of the above chondrogenic proteins is detected by Western Blot analysis (FIG. 9A). After repeating the experiment for three times, the statistical analysis results show that TFNA and TGF-β3 significantly promote the chondrogenesis of B bone marrow mesenchymal stem cells in vitro, especially in HTT and T+T treatment groups (FIG. 9E, FIG. 9F, FIG. 9G, and FIG. 9H).

The content of glycosaminoglycan in different groups is analyzed by Alyssin blue staining, as shown by FIG. 9I. Compared with the control group, more cells in HTT treatment group are dyed blue. In addition, the blue color (presented as different shades of black in the drawings attached in the present disclosure) of bone marrow mesenchymal stem cells treated by HTT is darker than that of other cells. The staining degree reflects that more glycosaminoglycans are synthesized in bone marrow mesenchymal stem cells treated with HTT.

Bone marrow mesenchymal stem cells are induced into cell spheres, and after 4 weeks of culture, the formation of type II collagen is detected by immunohistochemical staining, and the results show that the cell particles of mesenchymal stem cells in each group are dark brown (presented as different shades of black in the drawings attached in the present disclosure) as shown by FIG. 9J. The cell particles in the HTT-treated group have the darkest brown colour, suggesting that HTT-treated bone marrow mesenchymal stem cells have the highest expression of type II collagen and stronger cartilage-forming properties.

These results indicate that HTT significantly promotes the cartilage formation of bone marrow mesenchymal stem cells.

### 5. Effect of Bone Marrow Mesenchymal Stem Cells Ingested by HTT on OA Chondrocytes in Vitro

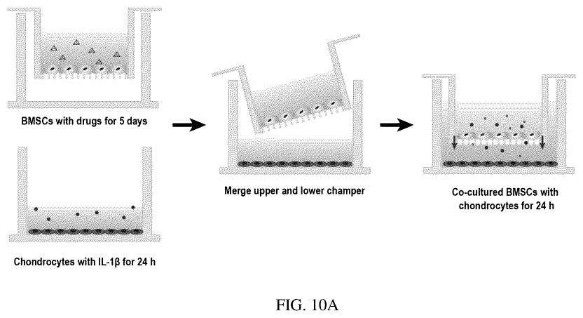

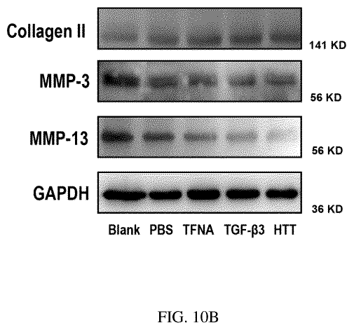

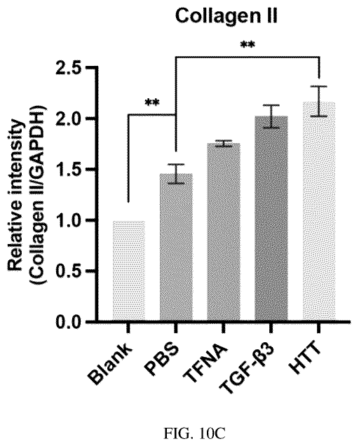

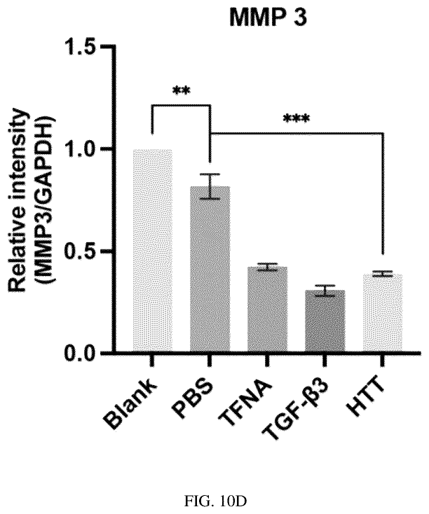

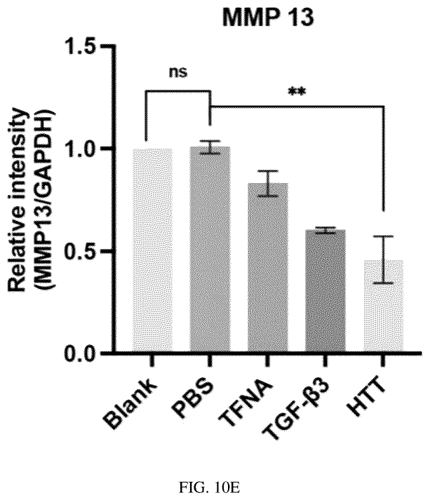

In order to study the interaction between bone marrow mesenchymal stem cells ingested by HTT and OA chondrocytes, the human chondrocytes are cultured with IL-1B (10 ng/mL) for 24 h to simulate OA chondrocytes in vitro. Bone marrow mesenchymal stem cells ingested by drugs are implanted into the upper cavity of transwell system, and chondrocytes induced by IL-1B are implanted into the lower cavity (see FIG. 10A). After 24 h incubation, chondrocytes are collected and their RNA and protein are isolated. RT-qPCR is used to detect the mRNA expression levels of specific matrix genes Collagen II, matrix metaloprotein-3 (MMP-3) and MMP-13. Western Blot is used to detect the protein expression levels of the above three genes.

The results, as shown in FIG. 10B, FIG. 10C, FIG. 10D, and FIG. 10E, suggest that HTT-treated bone marrow mesenchymal stem cells significantly increase the expression of type II collagen and decreases the levels of MMP-13 and MMP-3 in OA chondrocytes.

### Embodiment 3 Animal Model Experiment

### 1. Construction and Grouping of Animal Models

All animal experiment procedures are approved by the Institutional Animal Care and Use Committee (LAEC-2022-220) of Zhujiang Hospital of Southern Medical University. OA model is formed using 8-week-old male C57BL/6 mice with surgical destruction of the medial meniscus (DMM) (cited references: Glasson S S, Blanchet T J, Morris E A. The surgical destabilization of the medial meniscus (DMM) model of osteoarthritis in the 129/SvEv mouse. Osteoarthritis Cartilage. 2007; 15(9):1061-9.). Briefly, after anaesthesia, the medial capsule is incised on the right knee joint of mice, and the medial meniscus is removed. One week after operation, DMM mice are randomly divided into four treatment groups (n=12), including: 1) PBS; 2) TGF-β; 3) TFNA; and 4) HTT.

10 μL of different solutions (PBS, TGF-β3, TFNA, HTT) are slowly injected into the right knee of DMM mice every two weeks. The sham operation group is the normal control group, only the knee joint is exposed, and the ligament is not removed.

### 2. In Vivo Imaging

Mice are anaesthetised and injected intra-articularly with CY5-HTT 10 μL (1 μmol/L). The mice are then immediately placed in a bioluminescence imaging system (IVIS spectroscopy) to observe the retention of HTT in the joints of the mice, and fluorescence images at each time point of 0, 8 h, 1 d, 2 d, 3 d, and 5 d are collected for analysis. Mice injected with CY5-sDTC are used as a blank control.

### 3. Gait Analysis

Automatic gait analysis of walking mice is carried out by using catwalk XT (Noldus Information Technology BV, Lisburg, Virginia). All experiments are carried out and analyzed as previously reported. Briefly, the rats are trained to walk across the catwalk every day for 7 days prior to the operation. During the test, each rat is placed individually on the walkway and allowed to walk freely from one side of the walkway to the other. Light from enclosed fluorescent lamps is emitted within the glass panels and reflected entirely within. All data from the rat's paws is recorded by a high-speed colour video camera under the glass plate and connected to a computer running CatWalk v9.1 (Noldus Information Technology BV) software. Comparisons are made with respect to the left hind paw and the right hind paw for each run of each animal. The following gait parameters are calculated: right hind paw (RH)/left hind paw (LH) print area, RH/LH stress, and RH/LH swing speed.

### 4. Von Frey Test

Von Frey test is carried out on the right paw of mice one day before OA modeling and one day before each administration. Before each measurement, the mice are placed in a separate compartment on the metal mesh platform and adapted to the environment 30 minutes in advance. Von Frey fiber is measured by ascending stimulation method, where the Von Frey filaments are stimulated vertically upward in the middle of mouse feet, causing the filaments to bend for about 5 seconds, the same gram of filament is stimulated 5 times repeatedly, and the number of positive reactions is recorded. Positive responses are defined as toe contractions, licking, and fanning expansions that occur immediately after stimulus application and removal, excluding body contractions. Filaments recording three positive responses out of five measurements are labelled as pain thresholds.

### 5. Pathological Evaluation

Knee joint specimens of mice are fixed with 4% paraformaldehyde solution at room temperature for 48 hours, and then decalcified with EDTA decalcification solution on a vibrating table at 37° C. for 2 weeks, where the EDTA decalcification solution is replaced every 3 days. The knee joint specimens are dehydrated and embedded in paraffin, and sliced into 0.4 μm thick slices, stained with erythroxine O/solid green and toluidine blue to observe the degree of cartilage defect, OARSI score and cartilage area. The paraffin slices of knee joints are deparaffinised, hydrated, and antigenically repaired, and the primary antibodies against aggrecan, type II collagen, MMP-3, and MMP-13 (1:100) are incubated at 4° C. for 12 h. Subsequently, the corresponding secondary antibodies are incubated for 10 min at room temperature. After DAB staining, the nuclei are retained by hematoxylin and left to be observed.

TUNEL staining is used to confirm chondrocyte apoptosis in knee joints (Roche, Mannheim, Germany). Three slices from each knee joint of each group are taken for TUNEL histochemistry. DAPI counterstaining of chondrocyte nuclei is performed. The percentage of TUNEL positive nuclei in DAPI labeled nuclei of each individual is used to identify chondrocyte apoptosis and the average value of each treatment group.

### 6. Statistical Analysis

For quantitative statistics of immunohistochemical staining and TUNEL staining, semi-quantitative statistics of immunostaining and fluorescence intensity are performed using ImageJ 1.54b software (NIH, USA). Also, toluidine blue stained images are statistically measured for the area of dark hyaline cartilage region using ImageJ 1.54b software (NIH, U.S.A.) All experiments are repeated at least three times, and the results are expressed as mean (mean)±standard deviation (SD).

Prism 10.0 software (Graphpad, USA) is used for statistical analyses and statistical graphs plotting. For comparisons among multiple groups, the statistical method is one-way ANOVA; for comparisons between two groups, Student's t-test is used, and a statistical difference is considered when the p value is less than 0.05.

### 7. Results and Analysis

7.1 Effect of HTT on Ameliorating Pain Symptoms and Joint Function in DMM Model Mice

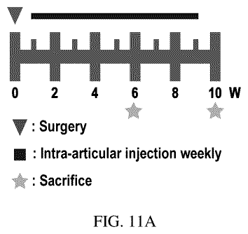

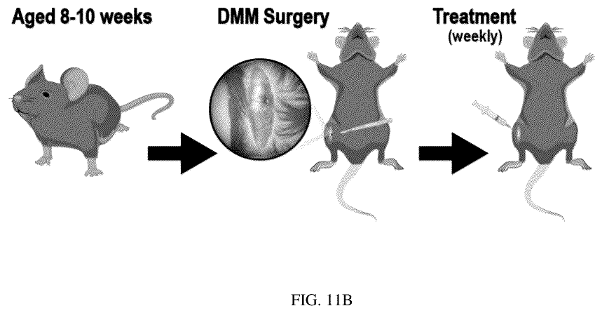

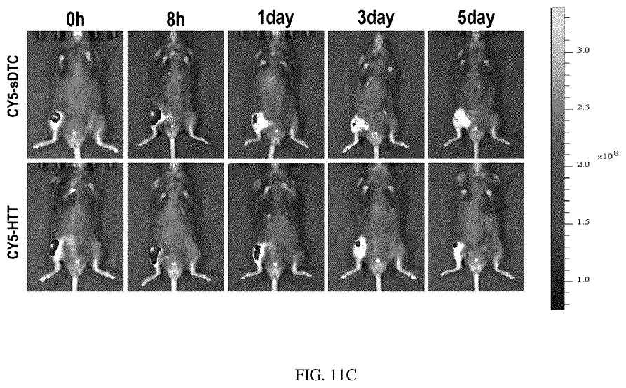

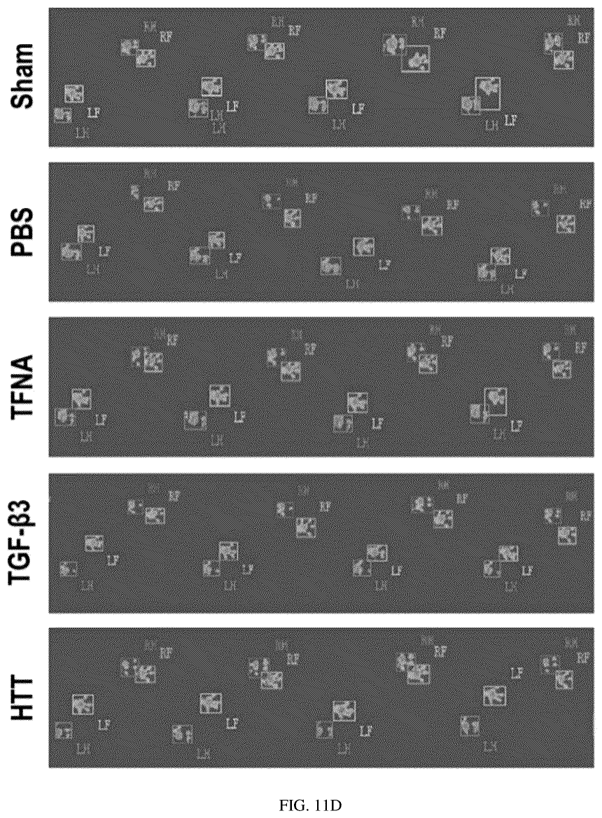

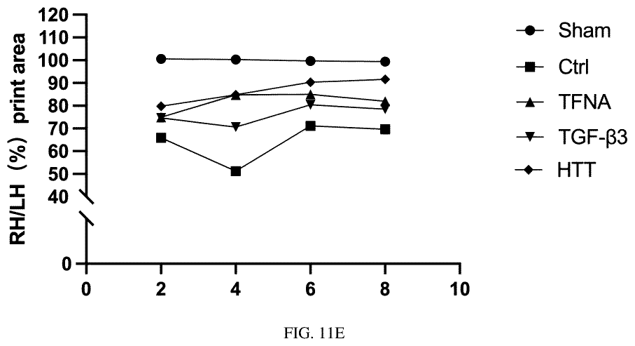

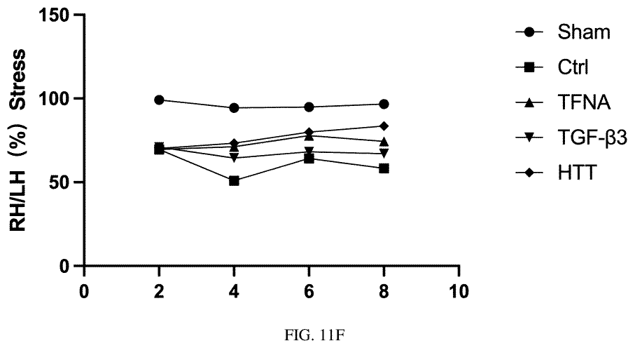

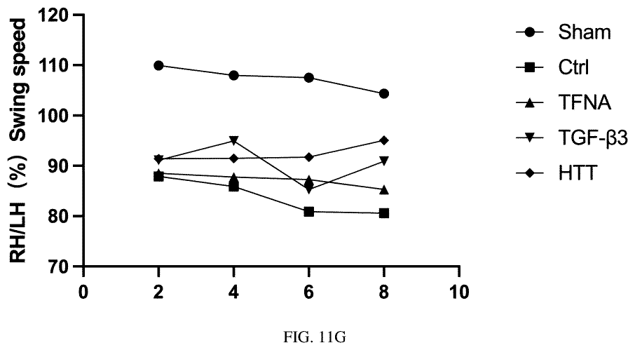

In the present disclosure, an OA model is established by performing DMM operation on 8-week-old mice, and drugs are injected into the joints every week one week after the operation (see FIG. 11A and FIG. 11B). The results show that compared with CY5-sDTC, the fluorescence of CY5-HTT stayed in mouse joints for a longer time, indicating that the residence time of HTT in mouse joints is longer than that of TGF-β3 alone (see FIG. 11C), and the decomposition and metabolism of HTT in joint cavity is slow; one of the reasons for this is that HTT is more targeted for cellular uptake and remains within the cell, thus avoiding being metabolized.

Gait analysis and Von Frey test are used to evaluate the improvement effect of HTT on pain and joint function in mice. The gait defects of mice are analyzed by gait analysis every two weeks, and the swing speed, contact area and paw stress of the affected and contralateral hind paws are compared (FIG. 11D). At the 2nd, 4th, 6th and 8th week after DMM (compared with sham operation), the contact area, stress and swing speed of the affected hind paw are significantly lower than those of the contralateral hind paw (see FIG. 11E, FIG. 11F, and FIG. 11G). The reduction is less significant in the HTT group, suggesting that HTT improves gait deficits as well as joint function most significantly.

Moreover, in the present disclosure, the Von Frey test is performed weekly on mice to analyse the right hind paw pain threshold. The results indicate that pain thresholds in mice are significantly lower after DMM operation and higher after treatment, with the HTT-treated group showing the most significant increase (see Table 4). These results suggest that HTT is more effective in improving pain symptoms and joint function in OA model mice than the administration of TFNA or TGF-β3 monotherapy.

TABLE 4
Results of Von Frey's experiment - Pain threshold of right hind paw in mice

| Group | Number | 0 | 1 w | 2 w | 3 w | 4 w | 5 w | 6 w | 7 w | 8 w | 9 w | 10 w |
|---|---|---|---|---|---|---|---|---|---|---|---|---|
| Sham | Sham01 | 4 | 0.4 | 0.4 | 1.4 | 2 | 4 | 4 | 2 | 2 | 2 | 2 |
| | Sham02 | 4 | 0.4 | 1 | 1 | 1.4 | 2 | 4 | 4 | 4 | 2 | 2 |
| | Sham03 | 4 | 0.6 | 1 | 1.4 | 4 | 4 | 4 | 4 | 2 | 4 | 4 |
| | Sham04 | 4 | 1.4 | 1.4 | 4 | 4 | 2 | 6 | 4 | 4 | 4 | 4 |
| | Sham05 | 6 | 1.4 | 1.4 | 1.4 | 1 | 2 | 2 | 4 | 4 | 4 | 4 |
| | Sham06 | 4 | 1.4 | 1.4 | 2 | 2 | 2 | 4 | 2 | 4 | 4 | 2 |
| | Average | 4.3 | 0.9 | 1.1 | 1.9 | 2.4 | 2.7 | 4.0 | 3.3 | 3.3 | 3.0 | 2.3 |
| PBS | PBS01 | 4 | 0.07 | 0.6 | 1 | 0.4 | 0.4 | 0.4 | 1 | 1.4 | 1 | 0.4 |
| | PBS02 | 4 | 0.4 | 1 | 1 | 0.6 | 1 | 1 | 0.6 | 0.6 | 1.4 | 0.6 |
| | PBS03 | 4 | 0.6 | 1 | 1 | 0.4 | 1 | 1 | 1 | 1 | 1.4 | 1 |
| | PBS04 | 6 | 0.6 | 0.6 | 1 | 1 | 1.4 | 1.4 | 0.6 | 0.4 | 1 | 1.4 |
| | PBS05 | 4 | 0.4 | 1 | 1.4 | 1.4 | 1 | 2 | 1.4 | 1.4 | 1 | 1 |
| | PBS06 | 4 | 0.4 | 1 | 1 | 1 | 1.4 | 1 | 1 | | 1 | 1 |
| | Average | 4.3 | 0.4 | 0.9 | 1.1 | 0.8 | 1.0 | 1.1 | 0.9 | 1.0 | 1.1 | 0.9 |
| TFNA | TFNA01 | 4 | 0.4 | 0.6 | 2 | 1 | 1.4 | 2 | 2 | 1 | 2 | 1.4 |
| | TFNA02 | 6 | 0.4 | 0.6 | 2 | 1 | 1.4 | 1.4 | 1.4 | 2 | 4 | 4 |
| | TFNA03 | 4 | 0.07 | 0.6 | 1.4 | 1.4 | 1.4 | 2 | 4 | 2 | 2 | 1 |
| | TFNA04 | 4 | 0.4 | 0.6 | 1.4 | 1 | 2 | 1.4 | 2 | 2 | 1 | 1.4 |
| | TFNA05 | 4 | 0.4 | 1.4 | 2 | 1.4 | 1 | 2 | 1.4 | 1.4 | 1.4 | 2 |
| | TFNA06 | 4 | 0.4 | 1 | 2 | 1.4 | 2 | 2 | 2 | 2 | 2 | 1.4 |
| | Average | 4.3 | 0.3 | 0.8 | 1.8 | 1.2 | 1.5 | 1.8 | 2.1 | 1.7 | 2.1 | 1.9 |
| TGF-β3 | TGF-B01 | 4 | 0.6 | 0.6 | 0.6 | 1 | 0.6 | 1.4 | 2 | 2 | 2 | 1.4 |
| | TGF-B02 | 4 | 0.07 | 0.4 | 1 | 1.4 | 1 | 2 | 2 | 2 | 2 | 1.4 |
| | TGF-B03 | 4 | 0.4 | 0.6 | 1 | 1.4 | 1.4 | 2 | 2 | 1 | 2 | 1.4 |
| | TGF-B04 | 6 | 0.6 | 1 | 1.4 | 1.4 | 1.4 | 2 | 2 | 1.4 | 3 | 1.4 |
| | TGF-B05 | 6 | 0.6 | 1 | 2 | 0.4 | 1.4 | 1.4 | 3 | 1.4 | 1.4 | 1.4 |
| | TGF-B06 | 4 | 0.4 | 0.6 | 1 | 1 | 1.4 | 1.4 | 2 | 1.4 | 2 | 1.4 |
| | Average | 4.7 | 0.4 | 0.7 | 1.2 | 1.1 | 1.2 | 1.7 | 2.2 | 1.5 | 2.1 | 1.4 |
| HTT | HTT01 | 4 | 0.4 | 0.6 | 0.6 | 1.4 | 4 | 2 | 2 | 1.4 | 2 | 2 |
| | HTT02 | 4 | 0.4 | 0.6 | 1 | 2 | 2 | 1.4 | 4 | 4 | 1.4 | 2 |
| | HTT03 | 6 | 0.6 | 0.6 | 1 | 1.4 | 2 | 2 | 2 | 2 | 4 | 2 |
| | HTT04 | 4 | 0.07 | 0.4 | 0.6 | 1.4 | 2 | 4 | 2 | 4 | 2 | 1.4 |
| | HTT05 | 6 | 0.4 | 1 | 0.4 | 2 | 1.4 | 4 | 2 | 1.4 | 2 | 2 |
| | HTT06 | 4 | 0.6 | 1 | 1 | 2 | 2 | 1.4 | 4 | 2 | 1.4 | 2 |
| | Average | 4.7 | 0.4 | 0.7 | 0.8 | 1.7 | 2.2 | 2.5 | 2.7 | 2.5 | 2.1 | 1.9 |

### 7.2 Protective Effect or OA Therapeutic Effect of HTT on Articular Cartilage in DMM Model Mice

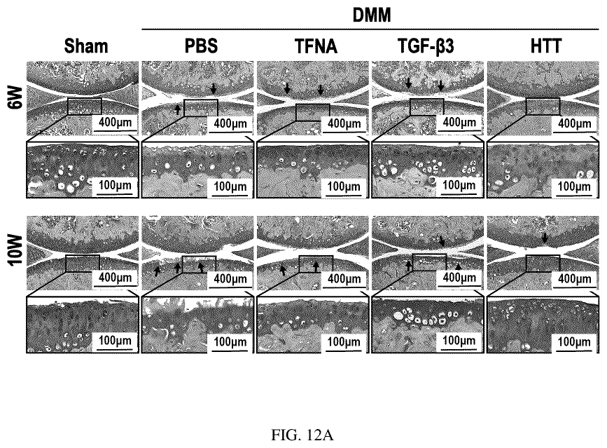

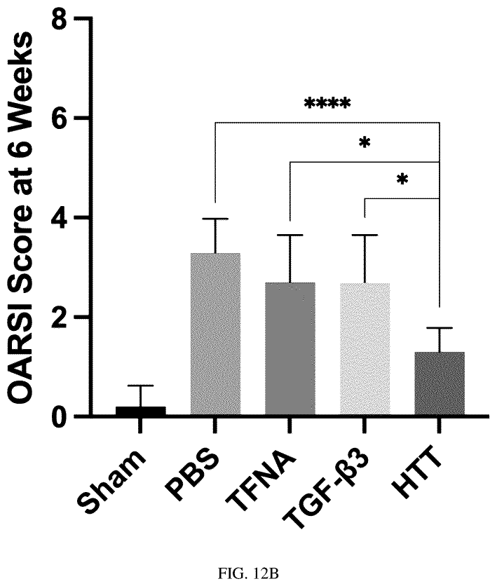

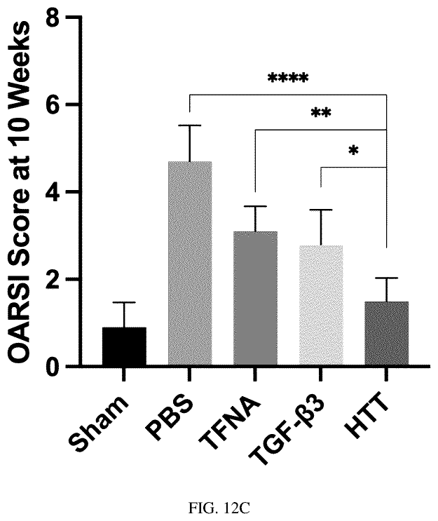

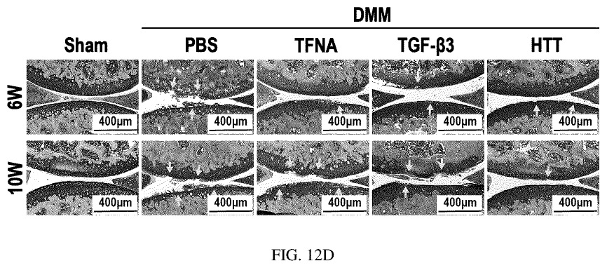

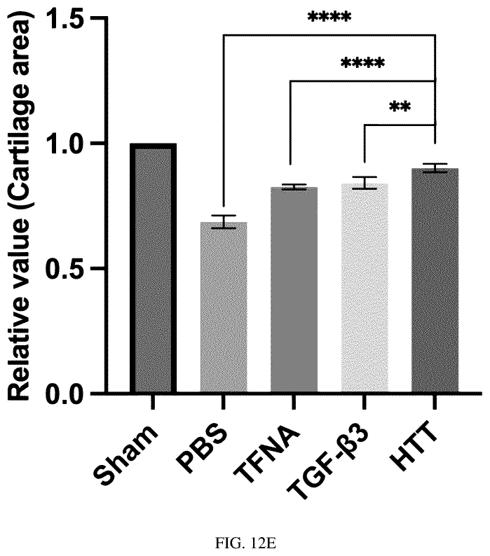

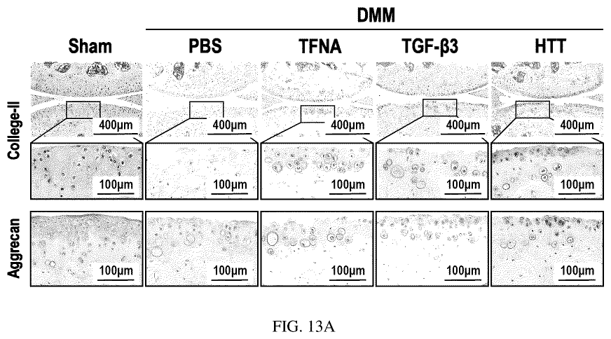

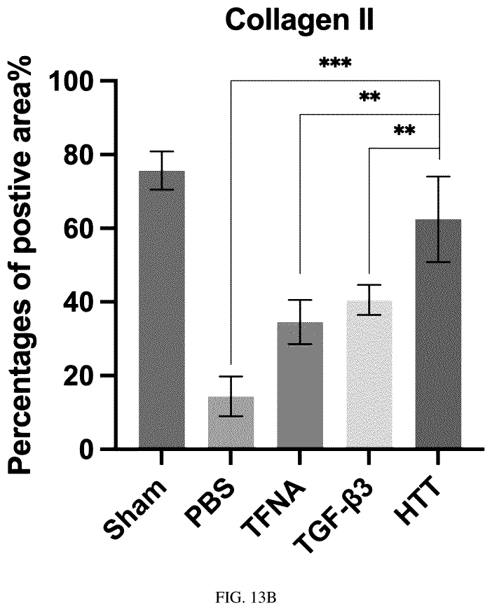

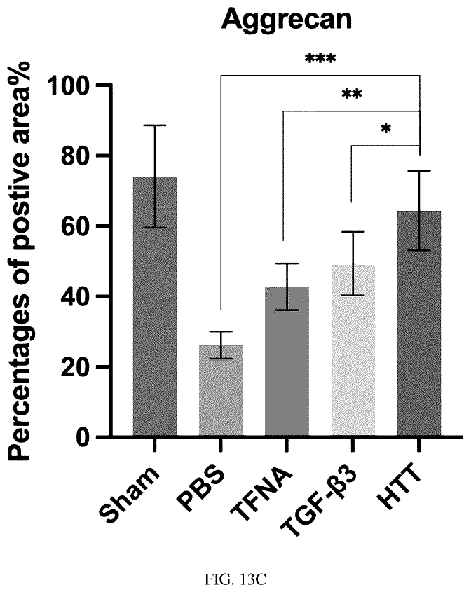

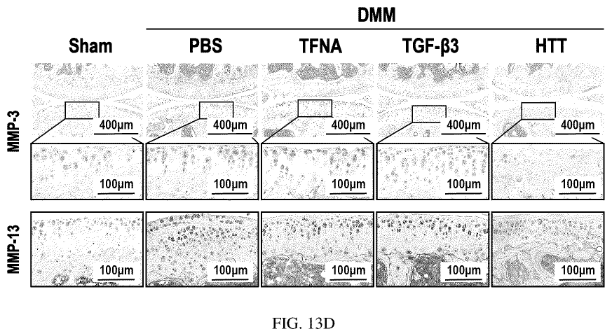

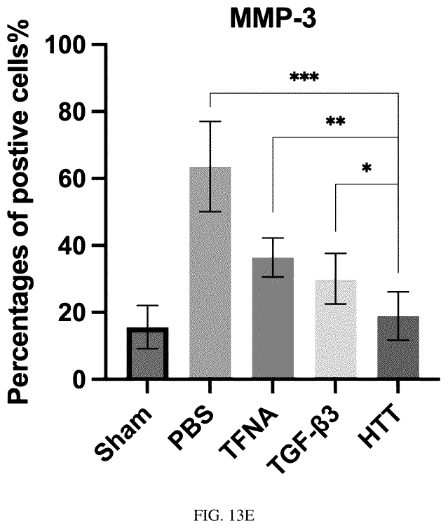

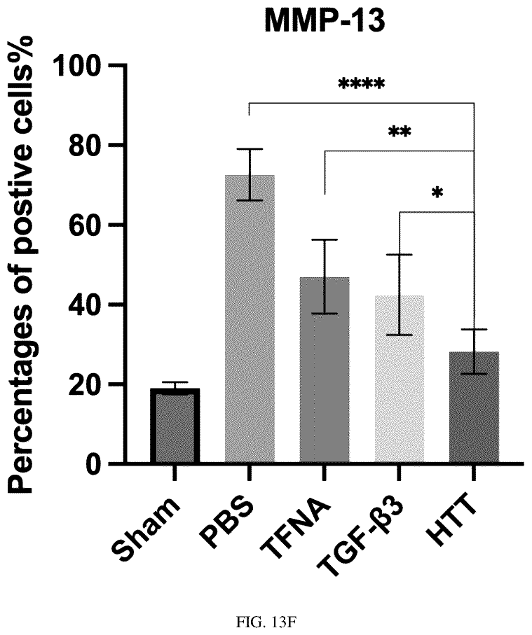

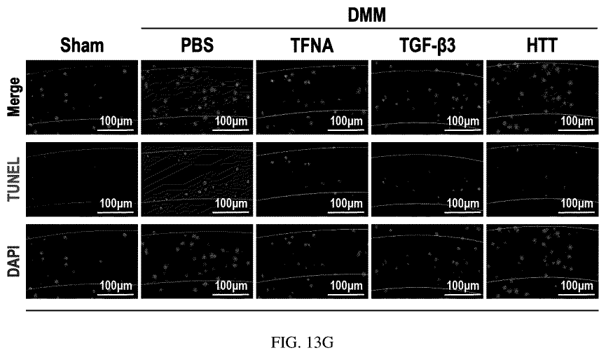

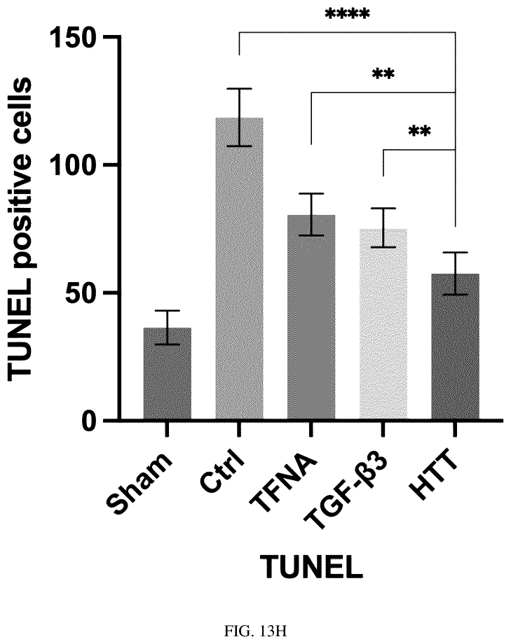

Six and ten weeks after operation, mice are killed in batches, and knee joint samples are collected. The joint defect and cartilage degeneration are evaluated by Safranine-O/Fast green staining (see FIG. 12A) and toluidine blue staining (see FIG. 12D), where the cartilage lesions are indicated by black arrows in FIG. 12A and white arrows in FIG. 12D, respectively, and the severity of the disease is evaluated by OARSI score (see FIG. 12B and FIG. 12C) and cartilage area (see FIG. 12E) (cited reference: Glasson S S, Chambers M G, Van Den Berg W B, et al. The OARSI histopathology initiative—recommendations for histological assessments of osteoarthritis in the mouse. Osteoarthritis Cartilage. 2010; 18 Suppl 3: S17-23.). For 10-week mouse joints, changes in cartilage biomarkers type II collagen and aggregated proteins in the cartilage matrix are studied by immunohistochemistry (IHC) (see FIG. 13A, FIG. 13B, and FIG. 13C). The positive areas for these two markers are significantly increased in the HTT-treated group compared to the PBS-treated group. Meanwhile, the positive rates of MMP-3 and MMP-13 are also reduced by the injection treatment, indicating that OA cartilage matrix degradation is alleviated by the treatment (see FIG. 13D, FIG. 13E, and FIG. 13F). The alleviation effect is most significant in the HTT-treated group. In addition, the results of TUNEL staining show that almost no apoptotic cells are observed on the surface and outer middle layer of articular cartilage in the sham operation group, while the apoptosis of chondrocytes is significantly increased in the PBS-treated group, and the apoptosis of chondrocytes in all treatment groups is reduced to different degrees. Among the groups, the lowest chondrocyte apoptosis rate is found in the HTT group (see FIG. 13G-FIG. 13H). In general, HTT provides the strongest protection of articular cartilage, effectively protecting against joint wear and delaying joint degeneration.

The above-mentioned embodiments only describe the preferred mode of the present disclosure, and do not limit the scope of the present disclosure. Under the premise of not departing from the design spirit of the present disclosure, various modifications and improvements made by ordinary technicians in the field to the technical scheme of the present disclosure shall fall within the protection scope determined by the claims of the present disclosure.

What is claimed is:

1. A tetrahedral framework nucleic acid modified by aptamer and transforming growth factor β3, wherein the tetrahedral framework nucleic acid combines transforming growth factor β3 through disulfide bonds; sequences of four DNA single strands of the tetrahedral framework nucleic acid are shown in SEQ ID NO: 1-4, one of the four DNA single strands of the tetrahedral framework nucleic acid also comprises an extended aptamer sequence, and the extended aptamer sequence is shown in SEQ ID NO: 5.

2. The tetrahedral framework nucleic acid modified by the aptamer and the transforming growth factor β3 according to claim 1, wherein the tetrahedral framework nucleic acid is a sulfhydryl modified tetrahedral framework nucleic acid.

3. A preparation method of the tetrahedral framework nucleic acid modified by the aptamer and the transforming growth factor β3 according to claim 1, comprising following steps:
assembling four DNA single strands by self-assembly to synthesize a tetrahedral framework nucleic acid;
subjecting the transforming factor β3 and N-succinimidyl-3-(2-pyridyl dithio)propionate to a mixing reaction to obtain activated transforming growth factor β3; and
subjecting the tetrahedral framework nucleic acid and the activated transforming growth factor β3 to a mixing reaction to obtain the tetrahedral framework nucleic acid modified by the aptamer and the transforming growth factor β3.

4. The preparation method according to claim 3, wherein conditions for the self-assembly comprise: denaturation at 95 degrees Celsius for 10 minutes, and cooling at 4 degrees Celsius for 20-30 minutes.

5. The preparation method according to claim 3, wherein a volume ratio of the transforming growth factor β3 to the N-succinimidyl-3-(2-pyridyl dithio)propionate is 1: (1-3); and
conditions for the mixing reaction of the transforming growth factor β3 to the N-succinimidyl-3-(2-pyridyl dithio)propionate comprise: reacting at 20-25 degrees Celsius for 2-3 hours.

6. The preparation method according to claim 3, wherein a volume ratio of the tetrahedral framework to the activated transforming growth factor β3 is (1-2): 1; and
conditions for the mixing reaction of the tetrahedral framework and the activated transforming growth factor β3 are: reacting at 20-25 degrees Celsius for 2-3 hours.
